(19) United States
(12) Patent Application Publication
(10) Pub. No.: US 2017/0156665 A1
(43) Pub. Date: Jun. 8, 2017

(54) IN VIVO MEASUREMENT OF CELL CONCENTRATIONS IN FLUID NEAR AN IMPLANTED MEDICAL DEVICE

(71) Applicant: DEF Medical Technologies, Inc., Philadelphia, PA (US)

(72) Inventors: Forrest Miller, Philadelphia, PA (US); Elsa Swanson, Philadelphia, PA (US); Emily Cen, Little Neck, NY (US); David Xing, Providence, RI (US)

(21) Appl. No.: 15/367,447

(22) Filed: Dec. 2, 2016

Related U.S. Application Data
(63) Continuation-in-part of application No. 14/956,813, filed on Dec. 27, 2016.
Application No. 14/956,813 is a continuation-in-part of application No. 14/953,394, filed on Jan. 26, 2016, now abandoned.

(51) Int. Cl. A61B 5/00

(52) U.S. Cl. CPC A61B 5/4851; A61B 5/0015; A61B 5/0082; A61B 5/7267

(57) ABSTRACT

To assist diagnosis of a post-operative condition related to an implanted medical device, such as an infection, inflammation, mechanical wear, and hemorrhaging, a monitoring device optically measures a concentration of a selected type of cells in a fluid surrounding the implanted medical device. The monitoring device comprises a light source and light sensor which are selected and calibrated, and placed in a relative spatial orientation with respect to each other, to detect light scattered by the selected type of cell in the fluid.

### CROSS-REFERENCE TO RELATED APPLICATIONS

This application claims the benefit of U.S. patent application Ser. No. 14/956,813, filed Dec. 2, 2015, and U.S. patent application Ser. No. 14/953,394, filed Jan. 26, 2016, the entire contents of which hereby are incorporated by reference herein and made part of this specification.

### BACKGROUND

Total joint arthroplasty (TJA) is a commonly performed surgical procedure in which a prosthetic joint is surgically implanted to replace an original joint. Total joint arthroplasty generally has high rates of success in alleviating pain and improving joint function, particularly in patients with advanced arthritis of the joint. There has been a steady rise in the number of TJA procedures due to a number of factors. There has been a concomitant rise in the number of revision surgeries performed post-primary TJA due to premature failure of the prosthetic joint.

The primary cause of premature failure of implantable medical devices, such as prosthetic joints, is periprosthetic infection. A periprosthetic infection is a condition in which a pathogen infects the body surrounding an implanted medical device.

To reduce the risk of such an infection, a health care provider commonly administers a prophylactic antibiotic to a patient at the time of surgery. Even with administration of antibiotics at the time of surgery, a post-operative periprosthetic infection still can occur. Most post-operative periprosthetic infections also can be treated with antibiotics if detected early. However, it is relatively difficult to detect an infection of an implanted medical device for several days after surgery due to a body's response to the surgery. Also, it is generally difficult to detect a bacterial infection of an implanted medical device in early stages of the infection, when symptoms are not yet present.

Physicians currently rely on indirect indications of infection, such as blood tests, self-report of pain, and observation of inflammation at the site of surgery. In the more specific case of joint replacement infections, pain is the predominant clinical symptom, and other symptoms may be absent or overlap with symptoms of other implant complications such as aseptic loosening. Other symptoms of infection which may present include swelling, erythema, local warmth, or drainage post-surgery. These indirect measurements generally are inconclusive until an infection is well-advanced, which may be two to three weeks after initial onset. A more direct measurement, such as cultures of tissue or fluid extracted from the site of the implanted medical device, generally is not performed until there are indirect indications suggestive of an infection.

Table 1 illustrates the most common microbes associated with periprosthetic infection:

TABLE 1

| Microorganism | % Infections |
|---|---|
| S. aureus | 55.8% |
| coagulase-negative | 17.6% |
| staphylococci, such as | |
| Staphylococcus | |
| epidermidis | |
| E. faecalis | 8.8% |
| E. coli | 8.8% |
| E. cloacae | 2.9% |
| K. pneumoniae | 2.9% |
| P. aeruginosa | 2.9% |

Periprosthetic joint infections (PJIs) in total joint arthroplasties are defined as meeting one or more of the following criteria: an abscess or sinus tract communicating with the joint space; a positive preoperative culture of aspirate; at least two positive intraoperative cultures of the same organism; or a positive culture in addition to either gross intracapsular purulence or abnormal histological findings.

These infections are classified into three types based on the timing of the infection: (i) acute postoperative (up to four weeks post-surgery) infection (Stage I); (ii) late chronic (over four weeks postoperative) infection (Stage II); and hematogenous (acute onset at a previously well-functioning prosthetic joint) infection (Stage III).

Acute postoperative (Stage I) infections are due to bacteria gaining access to the joint during or soon after the operation from either the skin or a draining wound. These infections usually show symptoms within a few days or weeks post-surgery.

Late chronic (Stage II) infections result from bacteria from air, surgical instruments, or the implant itself. The delay before symptoms present is due to the time organisms need to proliferate before becoming symptomatic.

In hematogenous (Stage III) infections, the organism is carried to the arthroplasty site via the bloodstream. In this scenario, a prior infection may enter the implant site. Alternatively, an acute surgical site infection may become systemic if the surgical site infection enters the bloodstream.

Among the classifications of periprosthetic infections, acute and hematogenous infections are more likely to be caused by more virulent pathogens such as Staphylococcus bacteria. When total knee replacements become infected, two of the three most predominant infectious organisms are strains of Staphylococcus bacteria: Staphylococcus aureus and Staphylococcus epidermidis. The clinical presentations of Staphylococcus vary depending on the location and severity of the infection; for example, boils, impetigo, cellulitis, and pus may develop on the skin, or blood poisoning can occur if the bacteria enter the bloodstream. If the patient develops toxic shock syndrome due to a Staphylococcus infection, the associated symptoms are fever, nausea, vomiting, rash, confusion, seizures, headaches, and muscle aches.

The clinical outcomes of Staphylococcus infections include death, irreversible damage to organ systems (such as endocarditis, pneumonia and osteomyelitis), and sepsis if the bacteria enter the human bloodstream. Septic arthritis also may develop, and if left untreated, the joint may be destroyed and the infection may spread to other parts of the body.

If bacteria enter the arthroplasty site, symptoms of infection typically present within days or weeks post-surgery. A biofilm may form within the first few weeks after surgery if the bacteria arrive at the implant prior to the arrival of benevolent human proteins and growth factors. Biofilms typically mature approximately two weeks after bacteria have arrived at the site of the implant. Approximately one week later, human leukocytes attach to the biofilms, but fail to phagocytose biofilm bacteria. It is therefore imperative to ensure that any bacterial infection is treated before the biofilm has the opportunity to form.

Before an infection can be treated, it must be diagnosed. The doctor first has to suspect infection before any tests are run to confirm the clinician's suspicion. These diagnostic tests include various imaging techniques (such as radiographs, radionuclide bone scans, and PET scans), and testing for biomarkers that are known to be associated with infection, such as white blood cell (WBC) count, erythrocyte sedimentation rate (ESR), and serum C-reactive protein (CRP) levels in blood tests, cultures, and assays.

Radionucleotide imaging can be used, but exact techniques vary. PET (positron emission tomography) imaging of indium-111 leukocytes is the best method currently used, but the specificity and sensitivity are variable.

Another technique is serology, which tests for elevated CRP or ESR levels, both measures of inflammation. The white blood cell (leukocyte) count, erythrocyte sedimentation rate (ESR) and plasma C-reactive protein (CRP) concentration in the blood of a patient become elevated as a result of the body's natural reaction to major surgery; however, these elevated levels also may occur if the patient has chronic inflammatory rheumatic disease. Accordingly, is imperative to establish a preoperative baseline for any measurements that will later be used for diagnostic purposes. CRP levels typically return to normal two to three weeks post-surgery; ESR may take 6 weeks or more to return to normal levels if an infection is not present. However, CPR and ESR levels will remain elevated in the presence of an infection. White blood cell count within the synovial fluid typically returns to normal levels as early as four days post-surgery if an infection is not present and will remain elevated in the presence of an infection. Such serology tests typically are performed at three months post-surgery, long after a biofilm may have formed.

Gram stains have low specificity and sensitivity, meaning the test has trouble distinguishing the Staphylococcus and Streptococcus bacteria that are likely to form biofilms.

Other examples of currently used methods include frozen sections of implant membranes which are similar in outcomes and limitations of joint fluid leukocyte counts but are much more involved and take longer. Newer tests, including tests for bacterial rRNA, have been found to be susceptible to false positives, as they are sensitive to dead bacteria as well as living ones. Joint fluid leukocyte counts are sensitive and specific, but standard tests are invasive, inconvenient and slow. Another newer test is for the proinflammatory peptide alpha-defensin, which must be performed at a specific laboratory; this test is highly accurate, but time-consuming as it must be performed remotely.

A serious drawback to methods currently used for detection of infection in implants is that soon after bacteria have colonized the surface of the implant, a biofilm begins to form. Studies have shown that surface characteristics of the prosthetic, such as roughness, hydrophobicity, and the lack of any antimicrobial coatings may contribute to increased biofilm formation. Furthermore, proteins such as fibronectin promote adherence of bacteria to biomaterial surfaces. Thus, an infection at the site of the implant must be detected and treated before the bacteria are able to form a biofilm.

If an infection has been diagnosed, patients who are deemed unable to undergo a revision arthroplasty (e.g., because the risk of surgery outweighs the benefits of replacement) typically are given a six-week program of intravenous antibiotics. These patients will have extremely poor quality of life, as the antibiotics alone will not cure the infection at this stage, especially if a biofilm has formed at the infection site. Alternatively, an operative debridement can be performed to remove the infection and biofilm, and the prosthesis is retained. This type of treatment is used only for acute postoperative PJIs and hematogenous PPIs that have been identified early. Another alternative to a full revision is a resection arthroplasty; this procedure is reserved for patients that cannot undergo the more extensive full revision surgery as its functional results are poor compared to that of full revisions.

There are two types of full revision surgeries: single-stage exchange and two-stage exchange revisions. The single-stage exchange revision surgery consists of the use of an antibiotic-loaded cement along with surgical debridement and a postoperative 6-week (minimum) course of parenteral antibiotics. This technique works best for patients with an acute infection (Stages I & III). Otherwise, a two-stage exchange revision is generally preferred. In this revision surgery, the first stage consists of removing all infected tissues and hardware and inserting an antibiotic-loaded spacer.

Such revision surgeries are often required because a patient does not present with clinical symptoms indicative of an infection. Thus, the infection is not diagnosed until it is too late for oral or intravenous antibiotic treatment. Even if the patient presents with symptoms, the infection often is diagnosed too late, or by the time test results to confirm the diagnosis return, it is too late for conservative retention treatment and extreme measures such as revision surgery are required.

As a result, many post-operative periprosthetic infections go undiagnosed until the infection is severe, such as when a biofilm has formed in the implanted medical device. While in some cases it is possible to administer further antibiotics, suppression of severe infection around an implanted prosthetic joint can require up to six weeks or more of antibiotic treatment, significantly decreasing a patient's quality of life. In some cases, the infection is too severe for antibiotic treatment, or such treatment is otherwise ineffective, or the infection results in loosening of the implant at the bone-cement interface. In these cases, a highly invasive revision surgery is usually required. Such revision surgeries are expensive, require removal and replacement of the implanted medical device and further increase the risk of infection, scarring, and permanent damage to the surrounding tissue.

Accordingly, what is needed is a reliable method of detecting and reporting post-operative periprosthetic infection of an implanted medical device, such as a prosthetic joint, and particularly a total joint prosthetic such as a knee, while the implanted medical device can still be saved.

### SUMMARY

White blood cell count in a fluid surrounding an implanted medical device is a useful indicator of post-operative infection because, in the absence of infection, white blood cell count typically returns to pre-operative levels within several days. If an infection is present, the white blood cell count can either remain elevated, or begin to increase after initially decreasing, and becomes substantially elevated above both the pre-operative level and ordinary post-operative level as the infection increases.

The concentration of white blood cells in a fluid surrounding an implanted medical device can be optically measured in vivo using a monitoring device positioned in a location exposed to the fluid surrounding the implanted medical device. The monitoring device can be affixed to the implanted medical device. Where the implanted medical device is a prosthetic joint, the monitoring device can be attached to a non-articulating surface of the prosthetic joint that is near the fluid being monitored.

The monitoring device comprises a light source and light sensor. The light source emits light into the fluid for a period of time, such as a few seconds. The light is scattered by elements of the fluid, such as white blood cells. The light sensor detects the scattered light and generates an output signal indicative of an amount of detected light. The amount of detected light is dependent upon amounts of elements in the fluid that scatter the light emitted by the light source, such as a white blood cell count. These amounts, or changes in these amounts over time, in turn are related to a likelihood of infection or other condition.

However, because many elements typically are present in fluids in the body, to measure concentration of a selected type of cell, such as a white blood cell, the light source and light sensor are selected and calibrated to the selected type of cell. The light source emits a narrow band of wavelengths of light which is approximately the same as a band of wavelengths of light which the selected type of cell reflects. Generally speaking, the band of wavelengths corresponds to the visible color of the type of cell, or the opposite color on the color wheel of the wavelength most absorbed by the type of cell. For white blood cells, this band is approximately 570 to 605 nm, as these cells are yellow-white. A light emitting diode that emits a narrow band of wavelengths of light around a nominal wavelength, such as 580-600 nm, with substantial attenuation, e.g., ten times, outside of this band, is an example of a suitable light source.

The light sensor is selected based on the light source and the likely high and low concentrations of cells to be measured in the fluid. For white blood cell counts in synovial fluid, the white blood cell count may be between approximately 4,200 cells/μL for a normal post-operative condition, to 92,600 cells/μL, or higher in the presence of an infection. The light sensor is selected and configured such that the output signal of the light sensor has an active range in which the output signal varies substantially in response to detected light between an amount of detected light corresponding to a low concentration of the type of cell in the fluid and an amount of detected light corresponding to a high concentration of the type of cell in the fluid. Preferably, the light sensor has a substantially linear response to scattered light in this active range.

The light source and light sensor are placed in a corresponding electrical circuit to power and control them. This electrical circuit can be implemented on a circuit board which also defines the relative spatial orientation of the light source and the light sensor. The relative spatial orientation of the light source and light sensor can be defined with respect to directions associated with these devices. In particular, light emanates from the light source in primarily a first direction into the fluid. Also, the light sensor has a surface on which light is primarily detected; a second direction is normal to and away from this surface into the fluid. To optimize detection of scattered light from a selected type of cell in a fluid, and reduce the influence of measuring indirect attenuation of light, these two directions generally should point, i.e., the light source and detector generally should be pointing, into the fluid, and at the same area in the fluid (and thus at an angle about 90 degrees or less), rather than pointing generally towards each other (particularly where the angle between the two directions is greater than approximately 135° which would result in indirectly measuring attenuation rather than directly measuring scattering). In other words, an angle formed at a point where these two directions intersect should be greater than or equal to 0° and less than 90°. The light source and light sensor also are spaced apart from each other at a sufficient distance such that light does not directly emanate from the light source into the light sensor.

To enable such an electrical circuit with a light sensor and light source to make measurements in vivo, the electrical circuit can be affixed to, or integrated directly into, an implanted medical device. Using a flexible circuit board for the electrical circuit allows the monitoring device to be mounted to a curved surface, such as those typically found on a prosthetic implant. The monitoring device is positioned on the implanted medical device to provide access to the fluid being measured without compromising function of the implanted medical device. For implanted medical devices that have moving parts, such as a prosthetic joint, the monitoring device is positioned on a non-articulating surface where it does not interfere with movement around the joint or of the subcomponents of the prosthesis itself.

Prior to surgery in which the monitoring device is implanted along with the implanted medical device, the monitoring device also is coated with a suitable biocompatible, translucent, dielectric coating at least on a surface of the monitoring device exposed in the body of the subject in which the monitoring device is placed in vivo. Such encapsulation typically is performed when the monitoring device is adhered to the medical device to be implanted, though it can also be performed beforehand and adhered later. The coating is dielectric to protect the monitoring device from interference with its operation from the fluid being measured. The coating is translucent to allow light to be transmitted through the coating. The coating is biocompatible to ensure that the monitoring device does not adversely impact the patient. An example of such a coating, which also can be used as an adhesive to secure the monitoring device to the implanted medical device, is a chemical vapor deposited poly(p-xylylene) polymer.

The electrical circuit of the monitoring device also includes a control circuit and wireless transmission circuit to allow data corresponding to the output signal of the light sensor to be generated and transmitted to an external device. The control circuit activates the light source and light sensor for a period of time for single measurement to cause the light sensor to generate an output signal. The wireless transmitter is configured to establish a communication connection with an external device and to transmit data corresponding to the generated output signal to the external device over the communication connection.

To assist diagnosis of a post-operative infection of a prosthetic joint, such a monitoring device measures, in vivo, the concentration of white blood cells in synovial fluid around the prosthetic joint over a post-operative time period. Such measurements can be taken during the first several weeks post-surgery, and can provide an earlier indication of infection than reliance on patient self-reporting or other currently used tests. A pre-operative baseline white blood cell concentration can be obtained through other methods by a health care provider to further assist in interpreting results from the monitoring device to provide a diagnosis. Example time periods, post-surgery, in which measurements can be taken can include, but are not limited to: the first three weeks, the first two weeks, the first week, the first ten days, between 4 and 7 days, between 4 and 10 days, between 4 and 14 days, or between 4 and 21 days, the first 90 days, and throughout the useful lifetime of the implanted medical device. Measurements can be taken at least daily during these time periods, and can include one measurement, two measurements or three measurements per day, for example. A single highly elevated measurement can be indicative of an infection. A difference between two measurements, or a trend defined by two or more measurements or three or more measurements, can be indicative of an onset of infection. The control circuit and wireless transmission circuit can be programmed or controlled from an external device to take multiple measurements during the post-operative time period. Such measurements can also be taken throughout the useful lifespan of the implant to identify later stage systemic and acute onset infections, with either the same or a lower frequency sample rate.

The monitoring device also can be configured to communicate with a computer system for a variety of purposes. Data corresponding to measurements made by the monitoring device can be stored on a patient device, such as a smart phone or other computing device. These data can be transferred to an electronic health record (EHR) system. These data can be processed further to assist in diagnosing conditions. Such data also can be used to build and apply machine learning models.

Such a monitoring device also detects changes in the optical quality of fluid surrounding an implanted medical device, such as an increase in turbidity or opacity of the fluid. For example, increases in the turbidity or opacity of the fluid surrounding the implant may be indicative of the presence of infection due to the presence of high concentrations of white blood cells and/or bacterial cells, or changes in the opacity of the fluid that may be due to the presence of blood, and thus an increasing concentration of red blood cells, leaking into the area around the implanted medical device.

The light source and the light sensor can be selected to measure concentrations of other cell types. For example, red blood cells will scatter more light with a light source having a wavelength in the range of about 620-750 nm. An elevated level of red blood cells in a fluid surrounding an implanted medical device can be indicative of hemorrhaging. Changes in these levels over time can be indicative of an onset of a condition such as hemorrhaging. Thus, the monitoring device can be designed to assist diagnosis of other conditions, such as inflammation, mechanical wear, or hemorrhaging, near an implanted medical device.

Thus, this monitoring device optically measures, in vivo, a concentration of a selected type of cells in a fluid surrounding an implanted medical device to assist in diagnosis of post-operative conditions related to the implanted medical device.

### BRIEF DESCRIPTION OF THE DRAWINGS

The accompanying drawings, which are incorporated into and form a part of the specification, are only for the purpose of illustrating one or more example implementations and are not to be construed as limiting the invention. In the drawings:

FIG. 1 is a perspective view of a simplified illustration of a prosthetic knee.

FIG. 2 is a perspective, exploded view of the prosthetic knee illustrated in FIG. 1.

FIG. 3A is an illustrative diagram of a principle of operation of a monitoring device.

FIG. 3B is an illustrative diagram of a configuration of a light source and light sensor on a circuit board.

FIG. 4 is a graph illustrating qualitative changes, and differences, in concentration of white blood cells in synovial fluid post operation in the presence and in the absence of infection.

FIG. 5 is a schematic circuit diagram of an example implementation of an electro-optical circuit for a monitoring device.

FIG. 6 is a schematic circuit diagram of an example implementation of an electro-optical circuit for a monitoring device.

FIG. 7 illustrates data obtained from experiments optimizing LED and photodiode orientation.

FIG. 8 illustrates output voltage with respect to white blood cell concentration data from experiments with the device of FIG. 6.

FIG. 9 is a block diagram of an example computer system with which the monitoring device can be used.

FIG. 10 is a flowchart describing an example implementation of transferring data from the monitoring device to the patient device.

FIG. 11 is a flowchart describing an example implementation of processing data from the monitoring device into a value representing cell concentration.

FIG. 12 is a block diagram of a general purpose computer.

### DETAILED DESCRIPTION

To assist diagnosis of post-operative conditions related to an implanted medical device, such as an infection, inflammation, mechanical wear, and hemorrhaging, a monitoring device optically measures a concentration of a selected type of cells in a fluid surrounding the implanted medical device. The monitoring device comprises a light source and light sensor which are selected and calibrated to detect light scattered by the selected type of cell in the fluid, or to detect changes in the optical quality of the fluid surrounding the implanted medical device, such as an increase in turbidity or opacity of the fluid. For example, increases in the turbidity or opacity of the fluid surrounding the implant may be indicative of the presence of infection due to the presence of high concentrations of white blood cells and/or bacterial cells, or changes in the opacity of the fluid that may be due to the presence of blood, and thus an increasing concentration of red blood cells, leaking into the area around the implanted medical device.

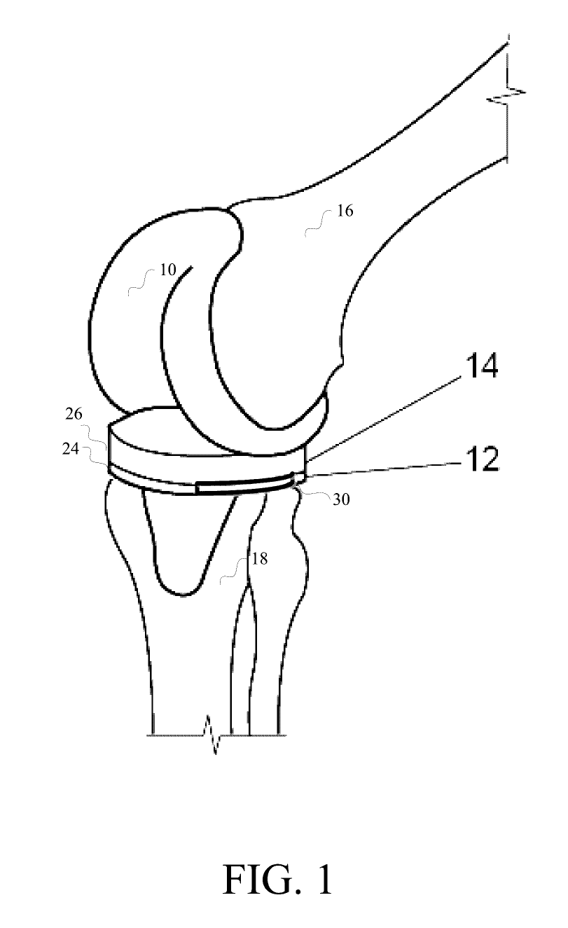

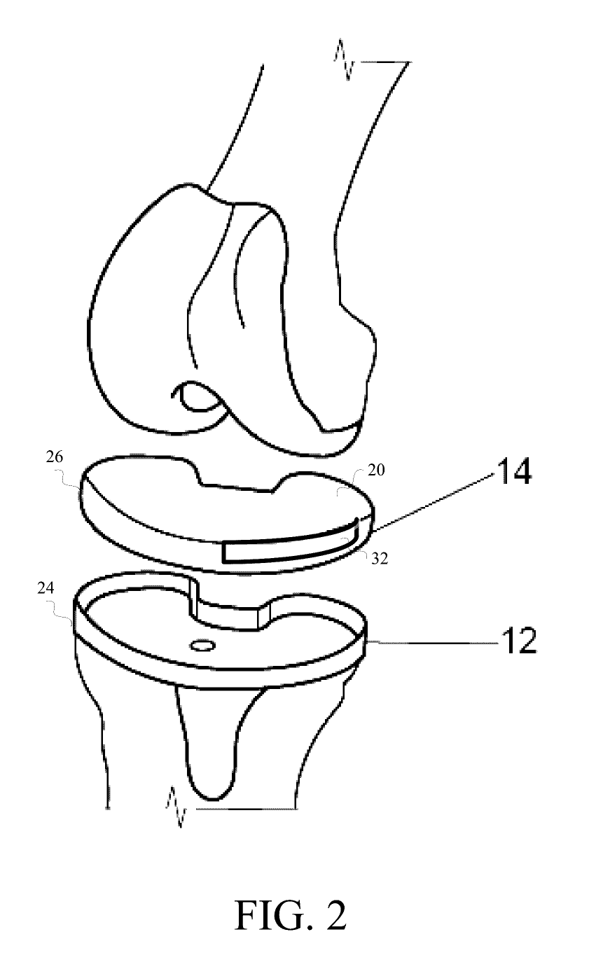

In the following description and associated drawings, an example implementation of such a monitoring device is provided in the context of measuring white blood cell count in synovial fluid surrounding a prosthetic joint, particularly a prosthetic human knee such as shown in FIGS. 1 and 2. The white blood cell count in synovial fluid has been found to be correlated with the presence (or absence) of an infection. Accordingly, by having a monitoring device, disposed on the implanted prosthetic joint, which measures white blood cell concentrations in the synovial fluid surrounding the prosthetic joint, an early indication of infection can be obtained. During the procedure to insert the prosthetic joint, the surgeon can extract synovial fluid and obtain a preoperative baseline white blood cell concentration to calibrate the output of the monitoring device. This pre-operative baseline can also be established through machine learning or other algorithm that incorporates variables such as age, sex, BMI, and any co-morbidity, drawing on a de-identified database of fluid measurements.

It should be understood that the invention is not limited to this example implementation, and that a monitoring device can measure concentrations of other types of cells, in other types of fluids, and in association with other types of implanted medical devices as described in more detail below.

Referring now to FIGS. 1 and 2, a simplified illustration of a prosthetic human knee incorporating such a monitoring device will now be described.

The knee is a joint in the human body, and is located where the thigh joins the lower leg. It consists of the lower part of the femur, the upper part of the tibia, and the patella or kneecap. A layer of cartilage surrounds the area where the bones meet to prevent bone wear and allow a smooth range of motion for the joint. Menisci between the tibia and femur absorb shock in the joint. The joint is surrounded by a synovial membrane, which is a thin layer of tissue that secretes lubricant which maintains low friction within the joint. There are four major ligaments in the knee: the anterior and posterior cruciate ligaments and the medial and lateral collateral ligaments.

When the cartilage wears away, the bones may start rubbing against each other, causing damage to the surface of the tibia and femur. In such cases, a knee arthroplasty can be conducted, inserting a prosthetic joint. In FIGS. 1 and 2, a surface 10 of the femur 16 is replaced with a biocompatible metal such as titanium. Similarly, a section of the tibia 18 is replaced with a biocompatible metal such as titanium, which forms a structure typically called a tibial tray 12. A polyethylene insert 14, also called the tibial insert, is mounted in the tibial tray 12. The surface 10 of the femur 16 and a surface 20 of the tibial insert 14 form the new joint. The surface 10 and surface 20 are referred to herein as the articulating surfaces of the prosthetic joint. This construction of the prosthetic knee allows full movement in the sagittal plane.

For a prosthetic joint, the monitoring device is positioned on a non-articulating lateral or medial surface of the prosthetic joint, and on a surface where ligaments and muscles and other tissues are very unlikely to come in contact with the sensors. The placement of the monitoring device with respect to the implanted medical device affects the measurement of cell concentration, as well as integrity of the implanted medical device. For a knee implant, the lateral tibial surfaces, such as the lateral surface 24 of the titanium tibial tray 12 or the lateral surface 26 of the tibial insert 14, are the preferred locations for measuring white blood cell count in the synovial fluid surrounding the joint. These lateral surfaces are non-articulating surfaces. The monitoring device can be incorporated into the titanium tibial tray or disposed on the surface of the polyethylene tibial insert. In this orientation the articulating surfaces of the prosthetic joint are not compromised by the monitoring device.

The monitoring device also can be positioned on the prosthetic joint such that, after the prosthetic joint is implanted, the monitoring device is directly adjacent to a synovial sac. In this orientation, the monitoring device is disposed in a location where it is readily perfused by synovial fluid which is constantly regenerated by synovial sacs. In such a location, changes in the synovial fluid which affect its optical properties are readily detected. Both the medial surface and the lateral surface satisfy this condition.

Referring again to FIGS. 1 and 2, the monitoring device for a prosthetic knee comprises an electro-optical circuit mounted on a lateral tibial surface of the implant. FIG. 1 shows the monitoring device incorporated into the tibial tray at 30. FIG. 2 shows the monitoring device disposed on the tibial insert at 32. The electro-optical circuit comprises a light source and a light detector, and other circuitry, an example of which is described in more detail below, mounted on a flexible printed circuit. By using a flexible printed circuit, the monitoring device can be shaped to conform to, and to be affixed to, the lateral tibial surface. A variety of techniques are available to affix such a flexible printed circuit to these surfaces. A thin layer of adhesive that forms covalent bonds when cured is preferable to provide a strong bond in response to sheer stresses that will likely impact the joint. Prior to surgery in which the monitoring device is implanted along with the implanted medical device, the monitoring device also is encapsulated with a biocompatible, translucent dielectric material. Such encapsulation typically is performed when the monitoring device is adhered to the medical device to be implanted, though it can also be performed beforehand and adhered later. A PARYLENE-brand polymer, or other polymers with similar properties, can be used for both purposes. PARYLENE-brand polymers, which are chemical vapor deposited poly(p-xylylene) polymers, can be used as both a biocompatible, translucent dielectric and an adhesive. It can be disposed in very strong, very thin layers, which is optimal for reducing shear stresses, due to the smaller area (thinner layer) on which those stresses can act.

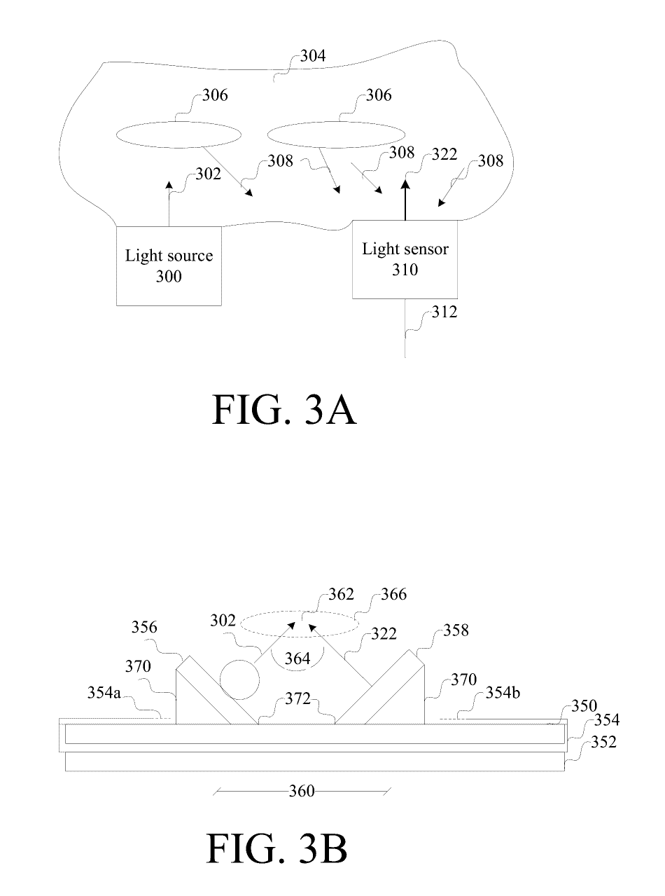

Turning now to FIG. 3A, a principal of operation embodied in the electro-optical circuit of the monitoring device will now be described. The monitoring device optically measures concentration of a type of cell in a fluid. The optical measurement uses a light source 300 that directs light 302 into the fluid 304. The light source is selected such that it generates a light having narrow band of wavelengths around a wavelength which is most reflected by the type of cell for which the concentration is being measured. As an example, considering white blood cell concentrations in synovial fluid, tests on such cells concluded that a light source that provides light in the band of 580-590 nm is suitable. Cells 306 present in the fluid 304 partially absorb, and partially reflect and scatter, the light 302, as indicated at 308. As a concentration of the white blood cells in the fluid increases, the amount of light that is scattered increases.

A light sensor 310, or photodetector, detects the scattered light 308 and generates an electrical signal 312, such as a voltage, as an output. The light sensor is selected and calibrated such that the output signal of the light sensor has an active range in which the output signal varies substantially in response to detected light between an amount of detected light corresponding to a low concentration of the type of cell in the fluid and an amount of detected light corresponding to a high concentration of the type of cell in the fluid. Also, the light sensor preferably has an output signal that varies linearly with concentration in this active range, in which case the electrical signal output by the light sensor is correlated with the concentration of cells in the fluid.

The light source and the light sensor also are arranged spatially, when the monitoring device is mounted on the implant, so as to optimize measurement of light scattered by the cells, in contrast to attenuation of light through the fluid. The light source has an associated direction (also indicated by 302) into the fluid in which light primarily emanates from the light source. While the light may emanate in a beam with multiple directions, the direction 302 is generally the direction of the light at the center of this beam, or the direction perpendicular to a center of a surface from which the light emanates. Similarly, the light sensor generally has a direction 322 into the fluid, normal to and away from a surface on which light is detected. These two directions 302, 304 form an angle at their point of intersection (see FIG. 3B). Experiments have shown that measurements by the light sensor are most correlated with concentration of white blood cell count when directions 302 and 322 are at about 45 degrees, and that such measurements have a greater dynamic range when this angle is orthogonal, i.e., 90 degrees.

To optimize detection of scattered light from a selected type of cell in a fluid, and reduce the influence of measuring indirect attenuation of light, these two directions generally should point, i.e., the light source and detector generally should be pointing, into the fluid and at the same area in the fluid (and thus at an angle about 90 degrees or less), rather than pointing generally towards each other (particularly where the angle between the two directions is greater than approximately 135° which would result in indirectly measuring attenuation rather than directly measuring scattering). In other words, the angle formed at a point where these two directions intersect should be greater than or equal to 0° and less than 90°. The light source and light sensor also are spaced apart from each other at a sufficient distance, and positioned at such an angle, such that light does not directly emanate from the light source into the light sensor. Given sizes of commercially available parts in the example implementations described below, such a distance is generally in around 5 mm as shown in FIG. 3B.

The relative spatial orientation of the light source and light sensor can be achieved through their placement on a circuit board, such as shown in FIG. 3B, which is an illustrative diagram and not shown to scale. In FIG. 3B, a circuit board 350 is shown attached to a surface of a medical device 352, such as with a very thin adhesive layer 354 (shown larger for purposes of illustration only). As described above, in an implementation using a material that is both an adhesive and a biocompatible, translucent dielectric, this layer 354 can encapsulate the circuit board and the components on it, as signified by the dashed lines 354a and 354b. The portion of the circuit board 350 shown in FIG. 3B includes a light source 356, such as an LED, and a light sensor 358, such as a photodiode. The light source 356 and the light sensor 358 are spaced apart by a distance 360, which will depend on the application and parts used. With the example circuit described below this distance 360 is about 5 mm and the total length of the circuit board section including the light source and light sensor is about 8 mm.

The light source 356 and the light sensor 358 are mounted on the circuit board so that they are angled towards each other. Direction 302 and direction 320 (as defined for FIG. 3A) intersect at a point 362. The angle 364 formed at this point is preferably less than 90 degrees. The directions 302 and 320 should be into the fluid and at the same area 366 in the fluid.

To make such a configuration, wedge shapes 370 can be inserted between the devices and the circuit board while maintaining surface area of solder pads. Similarly, the form factor of the devices can be changed so that they are angled, without sacrificing any surface area of its solder pads. The form factor of a device also can be changed, for example by cutting a side of the device as indicate at 372. A combination of these techniques can be used. As another alternative, if a flexible circuit board is used, then the circuit board can be adhered to the implanted medical device a way that creates the desired angle, either by bending and folding the circuit board into the desired position or by adhering additional material between the circuit board and the implanted medical device. When forming the circuit board, it also is desirable to limit the thickness of the circuit. When angling the light source and light sensor, changing their form factor of a device by current a side of the device, can reduce the overall thickness of the device. A device thickness of less than 3 mm, and preferably less than 2 mm, is desirable for attachment to an implanted medical device. A width of less than 5 mm and preferably less than 4 mm is suitable for articulating devices, such as prosthetic joints, to reduce the likelihood of interference with their motion.

For the purpose of detecting the presence of infection in the area of an implanted medical device such as a prosthetic joint, detecting an increase in the white blood cell count in fluid around the implanted medical device is a reliable and sensitive means to detect the infection before it becomes too advanced. Particularly, white blood cell count in the synovial fluid increases from about 4,200 cells/μL (microliter) to about 92,600 cells/μL, on average, in the case of infection. Most of the white blood cells that flood the synovial fluid in response to infection are neutrophils, which change the color and opacity of the synovial fluid, turning it from transparent to an opaque white-yellow. The concentration of white blood cells, particularly neutrophils, is causative of and highly correlated with the optical turbidity of the synovial fluid, therefore detection of changes in the optical properties of the synovial fluid provides a sensitive and specific method for early detection of infection.

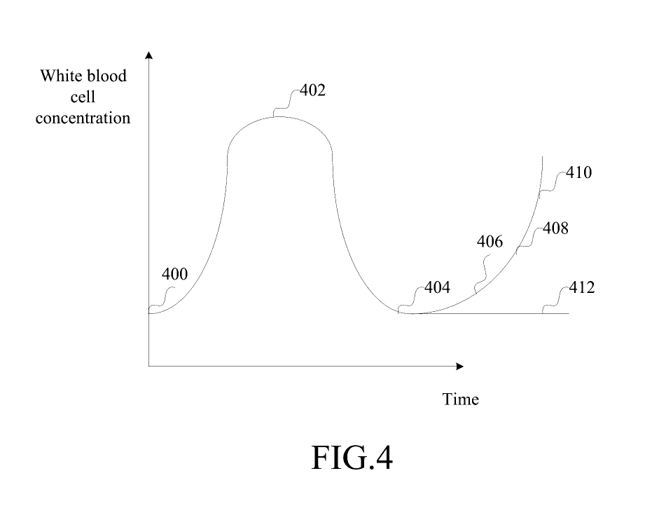

Referring to FIG. 4, a graph provides a qualitative illustration of white blood cell count (y axis) over time (x axis) around an operation. White blood cell count in synovial fluid increases from a pre-operative base condition 400, to an increased post-operative level 402. In the absence of infection, the white blood cell count typically returns to preoperative levels, as indicated at 404, within about four days after the surgery, and remains at this level as indicated at 412. In contrast, the white blood cell count in the synovial fluid increases sharply, often by orders of magnitude, if a bacterial infection has started to develop. Specifically, the white blood cell count may increase from approximately 4,200 cells/μL to 92,600 cells/μL, or higher. Thus, within the first four to fourteen days post-operation, if an infection develops, the white blood cell count will increase over time, as indicated at 406 and 408, and may reaching a highly elevated level 410. The white blood count generally persists at the elevated level throughout infection.

Accordingly, a monitoring device for detecting periprosthetic infection is designed to be operative to monitor white blood cell count in fluid around the implanted medical device for a period of at least several weeks, and up to years, post-surgery. Measurements from the monitoring device can be used to indicate a likelihood of a post-operative infection around the implanted medical device.

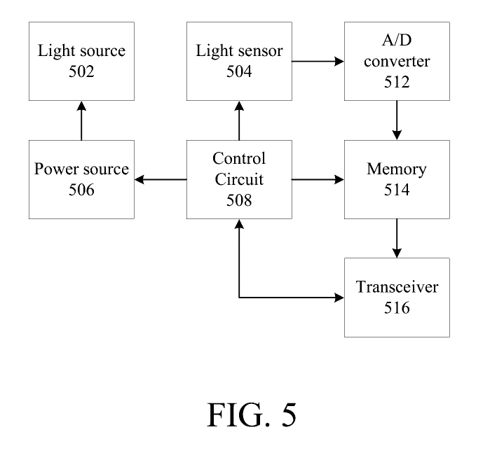

Turning now to FIG. 5, a schematic circuit diagram of an example implementation of the electro-optical circuit 500 of the monitoring device will now be described. In this example implementation, data representing the output voltage from the light sensor is captured into a memory for transmission from the monitoring device. In FIG. 5, the light source 502 and light sensor 504 are powered by a power source 506. A control circuit 508 controls whether the electro-optical circuit is operative. When operative, the light source 502 emits light and the light sensor 504 provides an output, as indicated at 510, based on light detected by the light sensor.

For the purposes of monitoring white blood cell counts in synovial fluid around prosthetic joints, the light source 502 can be implemented using a light emitting diode having a wavelength around 580 nm, corresponding to the apparent color of the cells (which represents the color that the cells reflect. For example, an LED that emits light within a 20 nm band, such as 580-600 nm, with significant attenuation outside of that band, e.g., 10 times, provides a good light source for this purpose. An example commercially available LED that can be used is the LY CNSM-FAGA-36-1-Z light emitting diode from OSRAM Opto Semiconductors, Inc., which has a nominal wavelength of 589 nm, and a band of wavelengths of about 583 nm to about 595 nm, and is characterized as yellow in color. The light sensor 504 can be implemented using a photodiode. An example commercially available photodiode that can be used is the BCS5030G1 photodiode from TDK Corporation of Japan, which has a nominal wavelength of 580 nm.

The output voltage of the light sensor 504 is generally in analog form, having a low voltage and a high voltage over an active range which is generally linear in response to different levels of light. This output voltage can be converted to digital form using an analog-to-digital converter 512. The digital representation of the output voltage can be stored in a memory 514.

The control circuit 508 can implement a timer so as to cause the circuit to be operative for a fixed period time for a single measurement. The period of time for a single measurement can be set experimentally so as to provide a reliable output signal from the light sensor. In one implementation, the period of time is several seconds, such as in the range of five to ten seconds. The control circuit 508 also can implement a timer to as to cause the circuit to periodically make such measurements. For the purpose of detecting infection by measuring white blood cell count, the frequency of such measurements generally is at least once daily, if not several measurements per day. As an alternative to a timer, such single measurements, and periodic initiation of such measurements, can be initiated by an external signal received by the control circuit 508.

Data gathered from the light sensor are stored in the memory 514, whether for a single measurement or for multiple measurements. The size of the memory 514 is dependent on the number of measurements for which data will be stored. The data then are transmitted from the monitoring device to an external device (described in more detail below) by a transceiver 516. The transceiver also can be used to receive external control signals and data. The transceiver can use a radio frequency signal to transmit data from the monitoring device to an external device. In one implementation, the transceiver 516 can be implemented in part using an integrated circuit that includes a programmable microcontroller, Bluetooth controller, and memory for data and programs for the microcontroller and Bluetooth controller. Such a chipset also can include the control circuit 508 and memory 514. An example integrated circuit for this purpose is SESUB-PAN-D14580 BLUETOOTH 4. 41 single mode module, which is a micromodule available from TDK Corporation of Japan, and which is a Semiconductor Embedded in SUBstrate (SESUB) embedded integrated circuit. Such a transceiver incorporates a Dialog Semiconductor DA14580 Bluetooth Low Energy (BTLE) controller, an ARM Cortex-MO 32-bit microcontroller, 32 kB of one-time programmable memory which can store custom computer programs, 84 kB ROM dedicated to the computer program code implementing the Bluetooth stack, 42 kB system SRAM, 8 kB retention SRAM, and a 16 MHz crystal. The transceiver can be configured to transmit the data at the time a single measurement is taken. Alternatively, the transceiver can be configured to transmit stored data from the memory in response to a request signal from an external device.

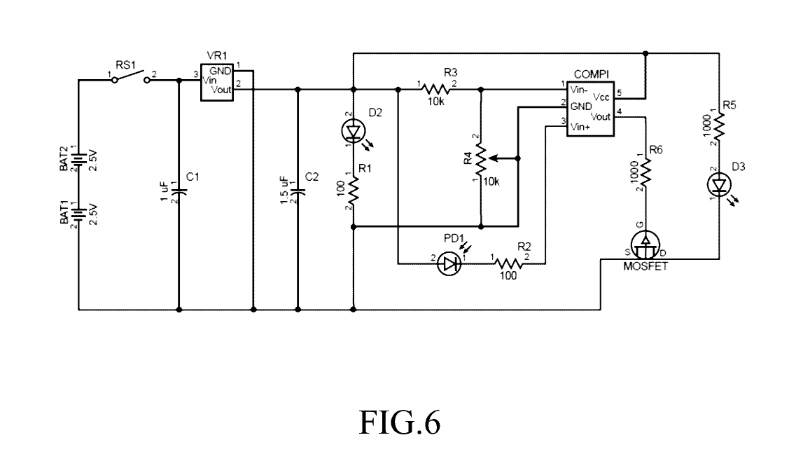

FIG. 6 is a schematic circuit diagram of another example implementation of a monitoring circuit. In this example implementation, the monitoring device generates an output indicative of whether the measured concentration of white blood cells has exceeded a threshold. In this implementation, two 2.5V batteries 600 power the apparatus, which is activated by the reed switch (RS1) 602. The voltage regulator (VR1) sets a constant 2.8V to the rest of the circuit to eliminate any unwanted fluctuations. The output of a photodiode 604 is fed into a first input of the comparator (COMP1) and a set threshold voltage, controlled by the potentiometer (R4) is fed into the other lead of the comparator. The comparator sends a high voltage to its V-out lead, connected to the gate of a MOSFET, when the photodiode voltage crosses the threshold. The MOSFET closes the infrared LED circuit, lighting it up, when the gate is high. In other words, the photodiode trips the infrared LED once the voltage output by the photodiode increases past a threshold value. This infrared signal is transmitted outside of the knee where it can be sensed by an external sensing device. Thus, in this example implementation, changes in the quality of the fluid above a certain level trigger a wireless notification device, which provides a single bit of data, alerting both patient and physician to a likelihood of an infection at the site of implantation.

In one implementation, the power source can be an inductive coupling powering system, such as a conducting coil, which, when subject to an external alternating electric field, supplies an induced current to the electro-optical circuit of the monitoring device. Such a coil can be disposed in a biocompatible sheath either distal or proximal to the monitoring device. In another implementation, the power source can be one or more batteries, as shown in FIG. 6.

The selection of the wavelength to be used for the light source to measure concentration of a type of cell in a fluid can be performed experimentally. In the case of white blood cells in synovial fluid, experiments were performed to confirm that mesenchymal stem cells (MSCs) are a reasonable substitute for white blood cells for these purposes. Next, spectrophotometer analysis was performed on six wells in a 96 well plate with varying concentrations of MSCs in synovial fluid, namely: 50,000 cells/μL, 25,000 cells/μL, 12,500 cells/μL, 6,250 cells/μL, 3,125 cells/μL, and 0 cells/μL. A full spectral sweep was performed to analyze the best wavelength to observe differences in cell concentration. Similar experiments can be performed for other types of cells in other types of fluids to identify a wavelength to measure concentration of that cell type in that fluid.

Relative orientation of the light source and light sensor also was determined experimentally. This relative spatial orientation affects the layout of the electro-optical circuit, and therefore affects alterations to the implanted medical device to which it is attached, as well as relative contributions of scattering and absorption characteristics to the total increase or attenuation of the output voltage of the light sensor.

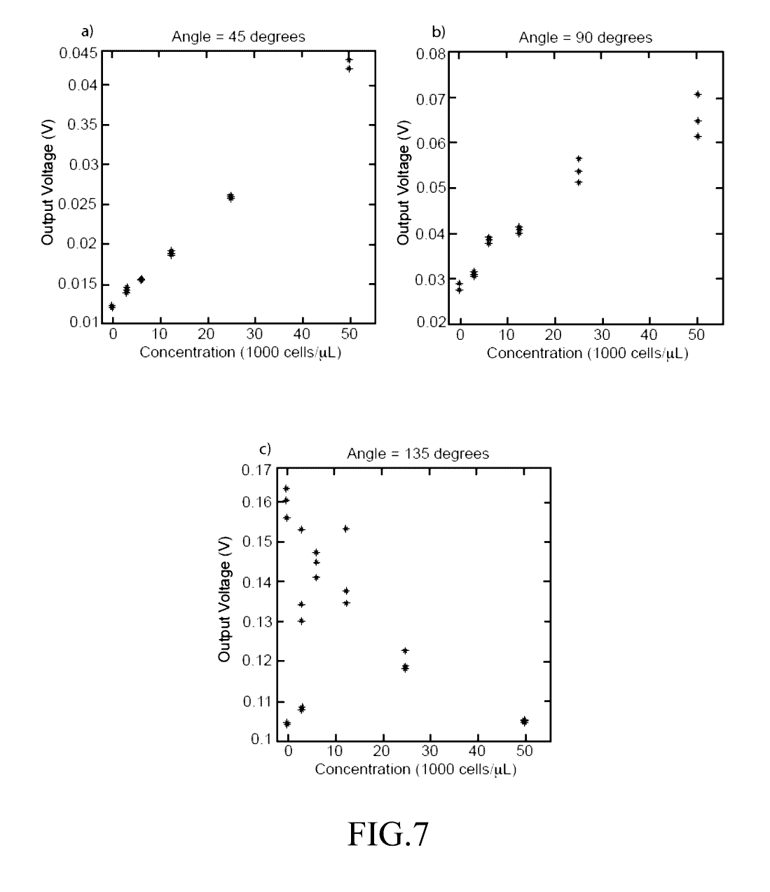

FIG. 7 illustrates data obtained from experiments optimizing the relative spatial orientation of a light source and light sensor, such as an LED and photodiode. A test circuit containing an LED in series with a 1.7 kOhm resistor and powered by a PSU supplying 5V was built. On the other side of the circuit, the photoresistor (Thorlabs FDS100) was added, with a bias voltage supplied by the PSU at 5V. The photoresistor was connected to an RC filter to remove noise (R=100 Ohms, C=1 microF), and also connected in series to a load resistor (1M Ohm) in order to measure output current. The LED was positioned so that the light beam would face the photoresistor surface. Using this test circuit, FIG. 7 is a graph illustrating results of output voltage vs. concentration from testing at three angles of relative orientation of an LED and photodiode: 45°, 90°, and 135°. There is a positive correlation between voltage and cell concentration at 45° and 90° and a negative correlation between the two at 135°. As seen in FIG. 7, results indicated that the circuit and components were sensitive enough to distinguish between infected levels and normal physiological levels of white blood cell count at 45° and 90° conformations.

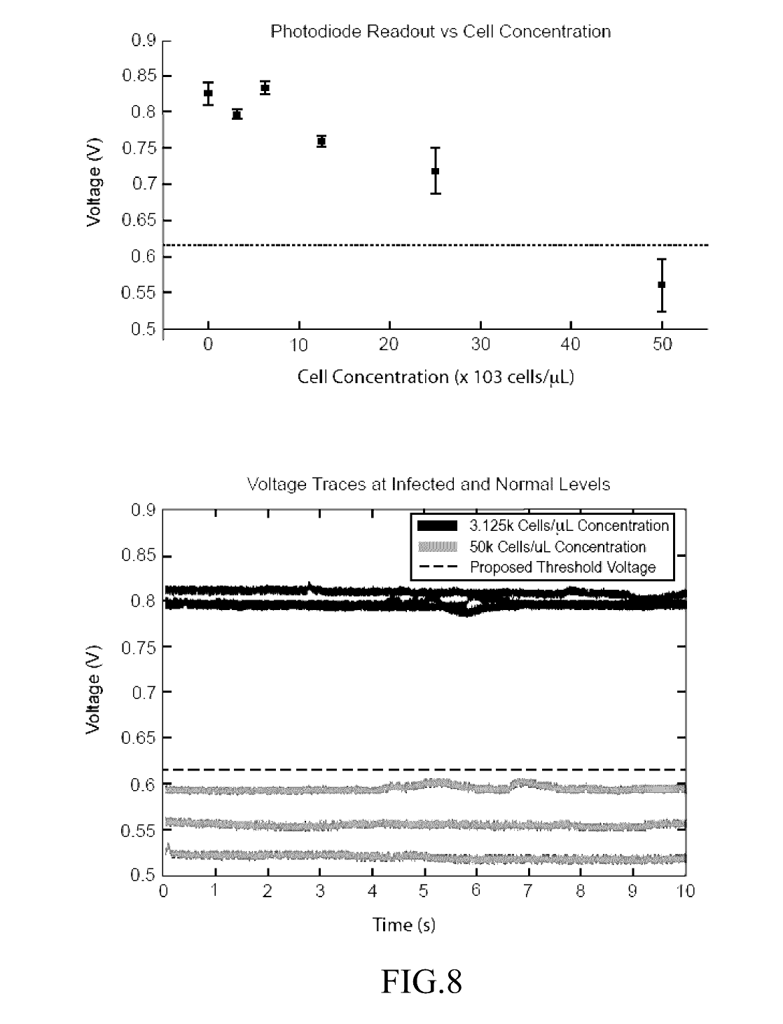

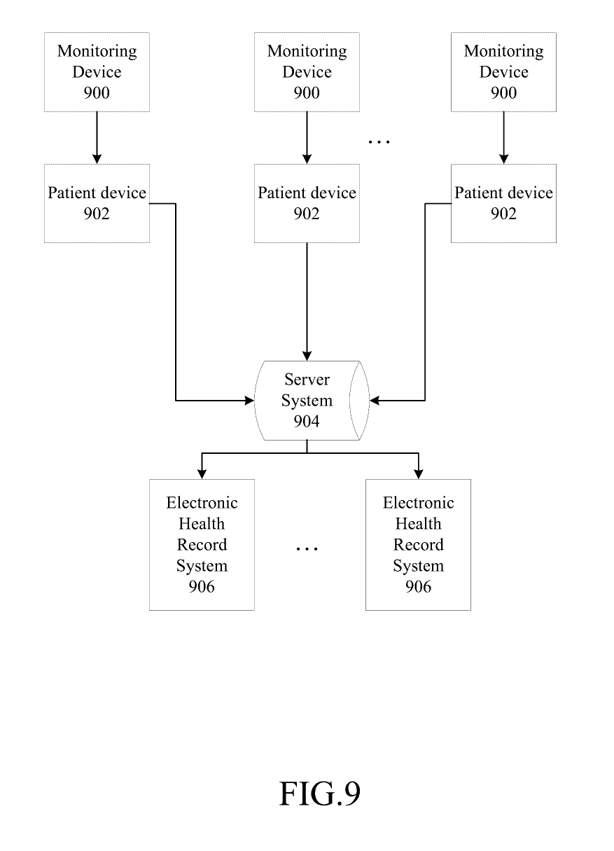

FIG. 8 is a graph illustrates output voltage vs white blood cell concentration data obtained in testing a device such as shown in FIG. 6 using the 90° relative spatial orientation. FIG. 9 illustrates output voltage vs concentration results for final testing. The LED and photodiode were oriented at 90 degrees and 5 mm apart from each other. The results show a ˜250 mV difference between infected and physiological levels, indicating that a clear distinction between the two cases could be made based on photodiode voltage alone. The voltage difference is negative because of this final embodiment's reverse voltage bias, which was implemented due to battery considerations.

There are several benefits from the monitoring device described above.

Significantly, optically measuring white blood cell concentration in vivo in the synovial fluid surrounding an implanted prosthetic joint allows infection to be detected prior to biofilm formation with a high specificity and sensitivity.

The components of the monitoring device are biocompatible, and therefore easily integrated into both an implant medical device and its environment in a body after being implanted. The monitoring device also includes a form of wireless communication that can transmit a signal indicative of the cell concentration. Because the monitoring device is integrated into the implanted medical device, the monitoring device requires no additional work from the surgeon to implement.

The monitoring device can communicate through its wireless transceiver to a computer system for a variety of purposes. An example implementation of such a computer system is shown in FIG. 9. In FIG. 9, the computer system provides a way to store measurement data from the monitoring devices for multiple patients into their electronic health records systems.

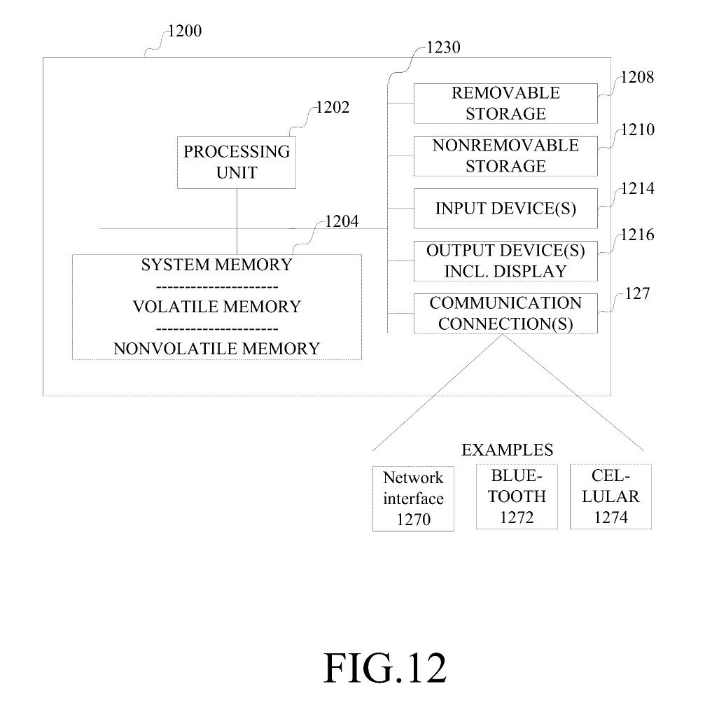

A monitoring device 900 for a patient connects to a patient device 902 owned by that patient. FIG. 9 shows multiple different monitoring devices 900, each connected to its own, different, patient device 902. The patient device 902 generally can be any general purpose computer (such as described below in connection with FIG. 12) owned by that patient and configured to communicate with the monitoring device 900. For example, if the patient device is a mobile telephone device, or a tablet computer, or a portable computer, or some other computer with a Bluetooth interface, the patient device can be paired with the monitoring device 900 to allow data from the monitoring device 900 to be transferred to the patient device.

In turn, the patient devices connect to a server computer 904. The server computer 904 is a general purpose computer, such as described below in connection with FIG. 12, and configured to operate as a server computer to interact with the patient devices 902, which act as client computers. The server computer 904 also is configured to operate as a HIPAA-compliant server to communicate confidential health information from the patient devices to their respective electronic health records systems 906. There may be multiple, different electronic health records systems 906, of which FIG. 9 shows at least two. The communication among the patient devices, server computer and electronic health records systems generally is performed over a computer network using a HIPAA-compliant protocol. An example of suitable, commercially available software for implementing a HIPAA-compliant server computer to connect to both patient devices and electronic health record systems is the CloudMine Connected Health Cloud software

In some implementations, the patient device 902 can be a computer controlled by the patient's healthcare provider which can connect with the patient's monitoring device given authorization from the patient. In some implementations, the monitoring device can provide data directly to the electronic health record system, or a server computer, used by the patient's health care provider.

In a patient device 902, computer program instructions are executed to transfer data from the monitoring device 900 and to store that data on the patient device. The control circuit and transceiver of the monitoring device are configured to coordinate with the patient device for this purpose. The data generally has a structure of a date and time stamp for when the data was captured by the monitoring device, and a stream of data samples for that measurement time. Computer program instructions also are executed to transmit that data to the server computer 904. In one of either the patient device 902 or the server computer 904, or the electronic health record system, the raw voltage data output from the monitoring device can be processed, by executing computer program instructions, into clinically meaningful data, such as a predicted concentration of a particular cell type. For example, the sample values for a given sample time can be averaged to provide a single, average sample value for that sample time. A mapping of sample values to concentration levels can be generated by preparing a statistical model from results of actual measurements. These results can originate, for example, from measuring multiple different, known samples with multiple monitoring devices. The results can originate from actual patient data, for example, by associating data from a patient's monitoring device with actual laboratory results for that patient.

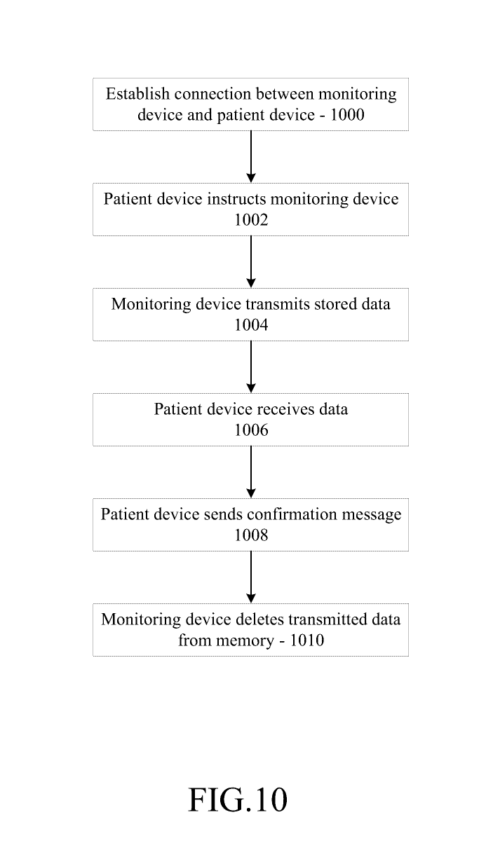

A process for transferring data from the monitoring device to the patient device will now be described in connection with FIG. 10.

In FIG. 10, the process is based on a connection being established 1000 between the monitoring device and the patient device. If the monitoring device communicates using Bluetooth or similar wireless transmission, a security protocol can be implemented so that the monitoring device for a patient can communicate only with a patient device for that patient or that patient's health care provider. For example, the monitoring device can be paired with the patient device prior to surgery.

After establishing a connection with the monitoring device, the patient device can instruct 1002 the monitoring device to transmit any stored data. In response to such an instruction, the monitoring device transmits 1004 stored data to the patient device. The protocol can include multiple steps to transmit the data to ensure proper receipt by the patient device. After the patient device receives 1006 the data, the patient device can send 1008 a confirmation message indicating the data has been received. If the monitoring device does not receive a confirmation message, in some implementations the monitoring device can attempt to retry the transmission. In response to a confirmation message, the monitoring device can delete 1010 the transmitted data from its local memory.

The process of interaction with the patient device also can include the patient device instructing the monitoring device to perform a measurement, and then to transmit the data after performing the measurement.

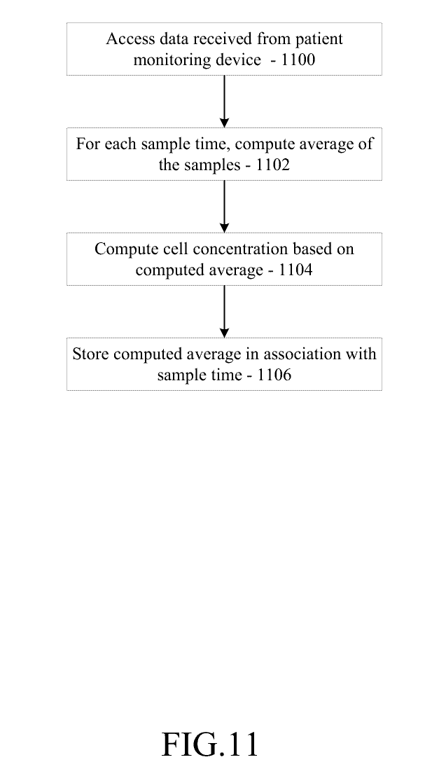

A process for converting data from the monitoring device into a measure of concentration of a cell type will now be described in connection with FIG. 11. The process begins with accessing 1100 the data received from the patient's monitoring device. This may process may be performed on the patient device, the server computer or the electronic health record system, and thus may involve transferring data to the computer which will process the data. For each sample time for which samples have been obtained, an average value is computed 1102. Given a statistical model that maps values from a monitoring device to a cell concentration, the computer computes 1104 a cell concentration for the computed average value. The computed average value can include an error range based on the statistical model. The computer then stores 1106 the computed cell concentration associated with a date and time corresponding to the sample time.

With such data stored, for multiple patients, in the electronic health record systems, or in the server computer, and if such data is associated with outcome data, further analyses can be performed by the server computer, electronic health record system, or other data processing system. Such analyses can include creation of machine learning models which can map data about concentrations of cells over time for multiple patients into candidate diagnoses, risk categories or the like. In general, features are extracted from the data about the concentrations of cells over time, such as differences between concentration levels at different points in time, and these features can be inputs to machine learning models along with other features extracted from patients health care records. As an example of how such machine learning models can be used, given multiple patients having a same condition, and having a same pattern over time occurring in their measured cell concentration data, such as white blood cell count, and having a same outcome for a given medical intervention, then another patient with the same condition can be identified as having a similar risk profile for the same output if their measured cell concentration data is observed to be following a similar pattern.

Having now described an example implementation, FIG. 12 illustrates an example of a computer with which components of the computer system of the foregoing description can be implemented. This is only one example of a computer and is not intended to suggest any limitation as to the scope of use or functionality of such a computer.

The computer can be any of a variety of general purpose or special purpose computing hardware configurations. Some examples of types of computers that can be used include, but are not limited to, personal computers, game consoles, set top boxes, hand-held or laptop devices (for example, media players, notebook computers, tablet computers, cellular phones including but not limited to “smart” phones, personal data assistants, voice recorders), server computers, multiprocessor systems, microprocessor-based systems, programmable consumer electronics, networked personal computers, minicomputers, mainframe computers, and distributed computing environments that include any of the above types of computers or devices, and the like.

With reference to FIG. 12, a computer 1200 includes a processing system comprising at least one processing unit 1202 and at least one memory 1204. The processing unit 1202 can include multiple processing devices; the memory 1204 can include multiple memory devices. A processing unit 1202 comprises a processor which is logic circuitry which responds to and processes instructions to provide the functions of the computer. A processing device can include one or more processing cores (not shown) that are multiple processors within the same logic circuitry that can operate independently of each other. Generally, one of the processing units in the computer is designated as a primary processor, typically called the central processing unit (CPU). One or more additional co-processing units 1220, such as a graphics processing unit (GPU), also can be present in the computer. A co-processing unit comprises a processor that performs operations that supplement the central processing unit, such as but not limited to graphics operations and signal processing operations.

The memory 1204 may include volatile computer storage devices (such as dynamic random access memory (DRAM) or other random access memory device), and non-volatile computer storage devices (such as a read-only memory, flash memory, and the like) or some combination of the two. A nonvolatile computer storage device is a computer storage device whose contents are not lost when power is removed. Other computer storage devices, such as dedicated memory or registers, also can be present in the one or more processors. The computer 1200 can include additional computer storage devices (whether removable or non-removable) such as, but not limited to, magnetically-recorded or optically-recorded disks or tape. Such additional computer storage devices are illustrated in FIG. 1 by removable storage device 1208 and non-removable storage device 1210. Such computer storage devices 1208 and 1210 typically are nonvolatile storage devices. The various components in FIG. 12 are generally interconnected by an interconnection mechanism, such as one or more buses 1230.

A computer storage device is any device in which data can be stored in and retrieved from addressable physical storage locations by the computer by changing state of the device at the addressable physical storage location. A computer storage device thus can be a volatile or nonvolatile memory, or a removable or non-removable storage device. Memory 1204, removable storage 1208 and non-removable storage 1210 are all examples of computer storage devices. Some examples of computer storage devices are RAM, ROM, EEPROM, flash memory or other memory technology, CD-ROM, digital versatile disks (DVD) or other optically or magneto-optically recorded storage device, magnetic cassettes, magnetic tape, magnetic disk storage or other magnetic storage devices. Computer storage devices and communication media are distinct categories, and both are distinct from signals propagating over communication media.

Computer 1200 may also include communications connection(s) 1212 that allow the computer to communicate with other devices over a communication medium. Communication media typically transmit computer program instructions, data structures, program modules or other data over a wired or wireless substance by propagating a modulated data signal such as a carrier wave or other transport mechanism over the substance. The term “modulated data signal” means a signal that has one or more of its characteristics set or changed in such a manner as to encode information in the signal. By way of example, and not limitation, communication media includes wired media, such as metal or other electrically conductive wire that propagates electrical signals or optical fibers that propagate optical signals, and wireless media, such as any non-wired communication media that allows propagation of signals, such as acoustic, electromagnetic, electrical, optical, infrared, radio frequency and other signals.

Communications connections 1212 are devices, such as a wired network interface, wireless network interface, radio frequency transceiver, e.g., cellular 1274, long term evolution (LTE) or Bluetooth 1272, etc., transceivers, navigation transceivers, e.g., global positioning system (GPS) or Global Navigation Satellite System (GLONASS), etc., transceivers, and network interface devices 1270, e.g., Ethernet, etc., or other device, that interface with communication media to transmit data over and receive data from signal propagated over the communication media.

The computer 1200 may have various input device(s) 1214 such as a pointer device, keyboard, touch-based input device, pen, camera, microphone, sensors, such as accelerometers, thermometers, light sensors and the like, and so on. The computer 1200 may have various output device(s) 1216 such as a display, speakers, and so on. Such devices are well known in the art and need not be discussed at length here. Various input and output devices can implement any interface technology that enables a user to interact with a device without constraints imposed by hand-operated and visually-dependent input devices such as mice, keyboards, remote controls, and the like.

The various computer storage devices 1208 and 1210, communication connections 1212, output devices 1216 and input devices 1214 can be integrated within a housing with the rest of the computer, or can be connected through various input/output interface devices on the computer, in which case the reference numbers 1208, 1210, 1212, 1214 and 1216 can indicate either the interface for connection to a device or the device itself as the case may be.

A computer generally includes an operating system, which is a computer program that, when executed, manages access, by other applications running on the computer, to the various resources of the computer. There may be multiple applications. The various resources include the memory, storage, input devices and output devices, such as display devices and input devices as shown in FIG. 1. To manage access to data stored in nonvolatile computer storage devices, the computer also generally includes a file system which maintains files of data. A file is a named logical construct which is defined and implemented by the file system to map a name and a sequence of logical records of data to the addressable physical locations on the computer storage device. Thus, the file system hides the physical locations of data from applications running on the computer, allowing applications to access data in a file using the name of the file and commands defined by the file system. A file system generally provides at least basic file operations such as creating a file, opening, a file, writing a file or its attributes, reading a file or its attributes, and closing a file.

The various modules, tools, or applications, and data structures and flowcharts of FIGS. 9-11, as well as any operating system, file system and applications on a computer in FIG. 12, can be implemented using one or more processing units of one or more computers with one or more computer programs processed by the one or more processing units.

A computer program includes computer-executable instructions and/or computer-interpreted instructions, such as program modules, which instructions are processed by one or more processing units in the computer. Generally, such instructions define routines, programs, objects, components, data structures, and so on, that, when processed by a processing unit, instruct or configure the computer to perform operations on data, or configure the computer to implement various components, modules or data structures.

While the specific implementations described above are for a prosthetic knee, the monitoring device may be used for detection of infection in other articulating implants such as artificial joints, in particular synovial joints, such as those located in the knees, hip, elbow, ankle, wrist, and vertebrae. Synovial joint implants may comprise, for example, hinge joints used in elbow joint replacement; saddle joints or condyloid joints used in wrist joint replacement; plane joints used between tarsal bones; or ball-and-socket joints used in hip replacement surgery. In each case, the monitoring device is disposed on a non-articulating surface of the implanted medical device, positioned such that the light source and photodetector are exposed to the synovial fluid, and on a surface where ligaments and muscles and other tissues are very unlikely to come in contact with the sensors. For example, for a prosthetic hip, the monitoring device can be positioned on the outside of the acetabular cup. The monitoring device also may be used for defection of infection in non-articulating implants such as arterial stents, ventricular shunts, fracture-fixation devices, implanted pacemaker-defibrillators or cosmetic implants. In each case, the monitoring device can be disposed on a surface of the implant and positioned such that the light source and photodetector are exposed to the surrounding fluid that may contain increasing levels of white blood cells, or other cells, if an infection is present.

The monitoring device also may be used in revision surgeries. For example, the monitoring device can be deployed on a lateral surface of a temporary spacer to monitor joint health throughout the exchange process. When appropriate, the temporary spacer is removed and a new prosthesis, which also may be equipped with the monitoring device, is implanted.

The monitoring device also may be used for detection of hemorrhaging around implanted medical devices. In particular, immediately post-surgery, the synovial fluid is perfused with red blood cells and has a similar composition to that of blood. The composition of the synovial fluid typically returns back to normal as the synovial sacs produce more synovial fluid. Typically, the red blood cells are largely resorbed in the days post-surgery. However, if hemorrhaging occurs, the concentration of red blood cells can increase or remain elevated. Thus, the concentration of red blood cells in synovial fluid, with respect to likelihood of hemorrhaging, has a qualitatively similar behavior over time post-surgery as the concentration of white blood cells in the synovial fluid, with respect to likelihood of infection. Also, by selecting a light source and light detector optimized for scattering light by red blood cells, the monitoring device can measure concentration of red blood cells in a similar manner. A single monitoring device could incorporate two sensors optimized, for example, for detection of white blood cells and red blood cells.

Thus, such a monitoring device can be used for detecting hemorrhaging around articulating implants such as artificial joints, in particular synovial joints, such as those located in the knees, hip, elbow, ankle, wrist, and vertebrae. In each case, the monitoring device is disposed on a non-articulating surface of the implanted medical device, positioned such that the light source and photodetector are exposed to the synovial fluid, and on a surface where ligaments and muscles and other tissues are very unlikely to come in contact with the sensors.

Also, such a monitoring device can be used for detecting hemorrhaging around non-articulating implants such as arterial stents, ventricular shunts, fracture-fixation devices, implanted pacemaker-defibrillators or cosmetic implants. In each case, the monitoring device can be disposed on a surface of the implant and positioned such that the light source and photodetector are exposed to the surrounding fluid that may contain increasing levels of red blood cells if hemorrhaging is occurring.

The monitoring device similar can be adapted to measure qualitative differences in cell concentrations for other types of cells by the selection of a light source and light detector optimized for scattering light by the type of cell of interest. The frequency of single measurements of a concentration of that type of cell, and the period of time over which such measurements can be made depend on the time period of interest in which the cell concentration can qualitatively change.

In some embodiments, the monitoring device may be coupled with therapeutic and/or prophylactic materials, e.g., an antibiotic material for the simultaneous detection and suppression of infection in implanted medical devices. In this type of embodiment, the monitoring device could trigger the release of an antibiotic through a comparator circuit similar to that shown in FIG. 7. A prophylactic, such as a drug-eluting prosthesis, would suppress the bacteria and the corresponding immune response, reducing the dynamic range of the monitoring device's output signal. However, it would also delay biofilm formation, facilitating sufficiently early treatment to avoid revision surgery.

In one aspect, a monitoring device for optical measurement, in vivo, of a concentration of a type of cell in a fluid around an implanted medical device in a body of a subject, comprises a light source and a light sensor. The light source has an output that emits a narrow band of wavelengths of light into the fluid, wherein the band of wavelengths corresponds to a visible color of the type of cell. The light sensor has a surface that detects light scattered by at least the cell in the fluid and an output that provides an output signal indicative of an amount of detected light, wherein the output signal of the light sensor has an active range in which the output signal varies substantially in response to detected light between an amount of detected light corresponding to a low concentration of the type of cell in the fluid and an amount of detected light corresponding to a high concentration of the type of cell in the fluid. An electrical circuit includes the light source and the light sensor and is formed in a circuit board, the circuit board providing a relative spatial orientation of the light source and the light sensor such that a first direction of light emanating from the light source and a second direction orthogonal to and away from the surface of the light sensor are into the fluid and at substantially a same area in the fluid. The electrical circuit further comprising a control circuit, the control circuit activating the light source and light sensor for a period of time for a single measurement to cause the light sensor to generate an output signal. The electrical circuit further comprises a wireless transmitter configured to establish a communication connection with an external device and to transmit data corresponding to the generated output signal to the external device over the communication connection.

In one aspect, a monitoring device for optical measurement, in vivo, of a concentration of a type of cell in a fluid around an implanted medical device in a body of a subject, comprises a light source and a light sensor. The light source and the light sensor are constructed and arranged such that the light sensor detects scattered light of the light source as scattered by at least the cell in the fluid and an output that provides an output signal indicative of an amount of detected scattered light. The light source can have a narrow band of wavelengths corresponding to a visible color of the type of cell. The light sensor can have an active range in which the output signal varies substantially in response to detected light between an amount of detected light corresponding to a low concentration of the type of cell in the fluid and an amount of detected light corresponding to a high concentration of the type of cell in the fluid. The relative spatial orientation of the light source and the light sensor be such that a first direction of light emanating from the light source and a second direction orthogonal to and away from the surface of the light sensor are into the fluid and at substantially a same area in the fluid.

In one aspect, a monitoring device for optical measurement, in vivo, of a concentration of a type of cell in a fluid around an implanted medical device in a body of a subject, comprises a light source and a light sensor. The light source emits light in a first direction into an area of the fluid. The light sensor has a surface with a second direction normal to and away from the surface into the area of the fluid, wherein an angle formed at a point of intersection of the first direction and the second direction is less than 90 degrees. The light source can have a narrow band of wavelengths corresponding to a visible color of the type of cell. The light sensor can have an active range in which the output signal varies substantially in response to detected light between an amount of detected light corresponding to a low concentration of the type of cell in the fluid and an amount of detected light corresponding to a high concentration of the type of cell in the fluid.

In one aspect, a monitoring device for optical measurement, in vivo, of a concentration of a type of cell in a fluid around an implanted medical device in a body of a subject, comprises a light source and a light sensor. The light source emits light in a first direction into an area of the fluid. The light sensor has a surface with a second direction normal to and away from the surface into the area of the fluid, wherein the first direction is away from the surface of the light sensor, whereby the light sensor detects light of the light source as scattered by cells in the fluid. The light source can have a narrow band of wavelengths corresponding to a visible color of the type of cell. The light sensor can have an active range in which the output signal varies substantially in response to detected light between an amount of detected light corresponding to a low concentration of the type of cell in the fluid and an amount of detected light corresponding to a high concentration of the type of cell in the fluid.

In one aspect, a method of optically measuring, in vivo, of a concentration of a type of cell in a fluid around an implanted medical device in a body of a subject, involves emitting light from a light source in a first direction into an area of the fluid. Light from the light source, as scattered by cells of the type of cell in the fluid, is detected by a light sensor directed, in a second direction, at the area of the fluid. The light source is directed away from the surface of the light sensor. The light source can have a narrow band of wavelengths corresponding to a visible color of the type of cell. The light sensor can have an active range in which the output signal varies substantially in response to detected light between an amount of detected light corresponding to a low concentration of the type of cell in the fluid and an amount of detected light corresponding to a high concentration of the type of cell in the fluid.

In any of the foregoing aspects, a control circuit can activate the light source and light sensor for a period of time for a single measurement to cause the light sensor to generate an output signal.

In any of the foregoing aspects, a wireless transmitter can establish a communication connection with an external device and to transmit data corresponding to the generated output signal to the external device over the communication connection.

In one aspect, a method for monitoring, in vivo, a concentration of a type of cell in a fluid around an implanted medical device in a body of a subject, wherein the implanted medical device includes a monitoring device that generates an output signal indicative of an indicative of an amount of detected light in the fluid in response to light emitted into the fluid, comprises causing the monitoring device to generate an output signal for a period of time for a first single measurement. A wireless communication connection is established between the monitoring device and an external device. The monitoring device is caused to transmit data corresponding to the output signal for the first single measurement to the external device. This method for monitoring can use a monitoring device or method of measuring in accordance with any of the aspects recited herein.

In one aspect, a system for monitoring, in vivo, a concentration of a type of cell in a fluid around an implanted medical device in a body of a subject, comprises a monitoring device in any of the aspects as recited herein and an external device configured to establish a wireless communication connection with the monitoring device and to cause the monitoring device to transmit data corresponding to the output signal for at least a first single measurement to the external device over the wireless communication connection.

In any of the foregoing aspects, an external device that connects to the monitoring device can be a general purpose computer executing an application to establish the wireless communication connection with the monitoring device. The external device can include a network interface for connection to a computer network and wherein the application transfers data received from the monitoring device to a server computer over the computer network. The external device can establish a connection between the monitoring device and the external device to cause the monitoring device to transmit data corresponding to the output signal for a second single measurement to the external device. The first single measurement and the second single measurement can be performed in a post-operative time period, after surgery to implant the implanted medical device.

In any of the foregoing aspects, in the active range of the output signal of the light sensor is substantially linear with respect to detected light.

In any of the foregoing aspects, the type of cell can be a white blood cell. For white blood cells, the light source can be a light emitting diode having a nominal wavelength in the range of 570-620 nm.

In any of the foregoing aspects, the type of cell can be a red blood cell. For red blood cells, the light source can be a light emitting diode having a nominal wavelength in the range of 620-750 nm. Multiple light sources can be used to detect multiple cell concentrations.

In any of the foregoing aspects, the implanted medical device can be a prosthetic joint. The monitoring device can be attached to a non-articulating surface of the prosthetic joint near the fluid. The fluid can be synovial fluid.

In any of the foregoing aspects, the circuit board can be a flexible printed circuit board.

In any of the foregoing aspects, the monitoring device can be coated with a biocompatible, translucent, dielectric polymer at least on a surface exposed in the body of the subject. The polymer can be a chemical vapor deposited poly(p-xylylene) polymer.

In any of the foregoing aspects, the first direction and the second direction form an angle, at a point of intersection, of less than 90°, i.e., the angle can be an acute angle or a right angle. The angle can be 45 degrees.

In any of the foregoing aspects, the monitoring device can have a power source. The power source can be a battery. The power source can include a conducting coil responsive to an external alternating electric field to generate an induced current to the electrical circuit.

In any of the foregoing aspects, data from the monitoring device for a single measurement can be transmitted to an external device. The data can be further transmitted from the external device over a computer network to an electronic health record system storing data for the subject. Such transmission can include transmitting the data from the external device over a computer network to a server computer.

In any of the foregoing aspects, a first single measurement and a second single measurement can be obtained. The first single measurement and the second single measurement can be performed in a post-operative time period, after surgery to implant the implanted medical device. The post-operative time period can be, after the surgery, the first three weeks, the first two weeks, the first week, the first ten days, between four and 10 days after surgery, between 4 and 14 days, between 4 and 21 days, between 4 and 7 days, 90 days, or throughout the useful lifetime of the implanted medical device.

In any of the foregoing aspects, multiple single measurements can be performed per day in a post-operative time period. A single measurement per day can be performed in the post-operative time period.

Having now described several example implementations, and principles and variations thereof, it should be understood that the subject matter defined in the appended claims is not necessarily limited to the specific implementations described above. The specific implementations described above are disclosed as examples only.

What is claimed is:

1. The monitoring device for optical measurement, in vivo, of a concentration of a type of cell in a fluid around an implanted medical device in a body of a subject, comprising:
a light source having an output that emits a narrow band of wavelengths of light into the fluid, wherein the band of wavelengths corresponds to a visible color of the type of cell;
a light sensor having a surface that detects light scattered by at least the cell in the fluid and an output that provides an output signal indicative of an amount of detected light, wherein the output signal of the light sensor has an active range in which the output signal varies substantially in response to detected light between an amount of detected light corresponding to a low concentration of the type of cell in the fluid and an amount of detected light corresponding to a high concentration of the type of cell in the fluid;
an electrical circuit including the light source and the light sensor and formed in a circuit board, the circuit board providing a relative spatial orientation of the light source and the light sensor such that a first direction of light emanating from the light source and a second direction orthogonal to and away from the surface of the light sensor are into the fluid and at substantially a same area in the fluid;
the electrical circuit further comprising a control circuit, the control circuit activating the light source and light sensor for a period of time for a single measurement to cause the light sensor to generate an output signal; and
the electrical circuit further comprising a wireless transmitter configured to establish a communication connection with an external device and to transmit data corresponding to the generated output signal to the external device over the communication connection.

2. The monitoring device of claim 1, wherein, in the active range of the output signal of the light sensor is substantially linear with respect to detected light.

3. The monitoring device of claim 1, wherein the type of cell is a white blood cell.

4. The monitoring device of claim 3, wherein the light source is a light emitting diode having a nominal wavelength in the range of 570-620 nm.

5. The monitoring device of claim 1, wherein the type of cell is a red blood cell.

6. The monitoring device of claim 5, wherein the light source is a light emitting diode having a nominal wavelength in the range of 620-750 nm.

7. The monitoring device of claim 1, wherein the implanted medical device is a prosthetic joint.

8. The monitoring device of claim 7, wherein the monitoring device is attached to a non-articulating surface of the prosthetic joint near the fluid.

9. The monitoring device of claim 8, wherein the fluid is synovial fluid.

10. The monitoring device of claim 1, wherein the circuit board is a flexible circuit board.

11. The monitoring device of claim 1, wherein the monitoring device is coated with a biocompatible, translucent, dielectric polymer at least on a surface exposed in the body of the subject.

12. The monitoring device of claim 11, wherein the polymer is a chemical vapor deposited poly(p-xylylene) polymer.

13. The monitoring device of claim 1, wherein the first direction and the second direction form an angle, at a point of intersection, of less than 90°.

14. The monitoring device of claim 13, wherein the angle is an acute angle or a right angle.

15. The monitoring device of claim 13, wherein the angle is a right angle.

16. The monitoring device of claim 13, wherein the angle is an acute angle.

17. The monitoring device of claim 1, further comprising a power source.

18. The monitoring device of claim 17, wherein the power source comprises a conducting coil responsive to an external alternating electric field to generate an induced current to the electrical circuit.

19. A method for monitoring, in vivo, a concentration of a type of cell in a fluid around an implanted medical device in a body of a subject, wherein the implanted medical device includes a monitoring device that generates an output signal indicative of an indicative of an amount of detected light in the fluid in response to light emitted into the fluid, the method comprising:
causing the monitoring device to generate an output signal for a period of time for a first single measurement;
establishing a wireless communication connection between the monitoring device and an external device;
causing the monitoring device to transmit data corresponding to the output signal for the first single measurement to the external device.

20. The method of claim 19 wherein the monitoring device comprises:
a light source having an output that emits the light having a narrow band of wavelengths into the fluid, wherein the band of wavelengths corresponds to a visible color of the type of cell;
a light sensor having a surface that detects light scattered by at least the cell in the fluid and an output that provides the output signal indicative of an amount of detected light, wherein the output signal of the light sensor has an active range in which the output signal varies substantially in response to detected light between an amount of detected light corresponding to a low concentration of the type of cell in the fluid and an amount of detected light corresponding to a high concentration of the type of cell in the fluid;
an electrical circuit including the light source and the light sensor and formed in a circuit board, the circuit board providing a relative spatial orientation of the light source and the light sensor such that a first direction of light emanating from the light source and a second direction orthogonal to and away from the surface of the light sensor are into the fluid and at substantially a same area in the fluid;
the electrical circuit further comprising a control circuit, the control circuit activating the light source and light sensor for a period of time for a single measurement to cause the light sensor to generate an output signal; and
the electrical circuit further comprising a wireless transmitter configured to establish a communication connection with an external device and to transmit data corresponding to the generated output signal to the external device over the communication connection.

21. The method of claim 19, wherein the monitoring device is coated with a biocompatible, translucent, dielectric polymer at least on a surface exposed in the body of the subject.

22. The method of claim 19, further comprising:
transmitting the data from the external device over a computer network to an electronic health record system storing data for the subject.

23. The method of claim 19, further comprising:
transmitting the data from the external device over a computer network to a server computer.

24. The method of claim 19, further comprising:
causing the monitoring device to generate an output signal for a period of time for a second single measurement;
establishing a connection between the monitoring device and an external device;
causing the wireless transmitter to transmit data corresponding to the output signal for the second single measurement to the external device.

25. The method of claim 24, wherein the first single measurement and the second single measurement are performed in a post-operative time period, after surgery to implant the implanted medical device.

26. The method of claim 25, wherein the post-operative time period comprises a first three weeks after the surgery.

27. The method of claim 26, wherein the post-operative time period is the first two weeks.

28. The method of claim 26, wherein the post-operative time period is the first week.

29. The method of claim 26, wherein the post-operative time period is the first ten days.

30. The method of claim 26, wherein the post-operative time period is between four and 10 days.

31. The method of claim 26, wherein the post-operative time period is between 4 and 14 days.

32. The method of claim 26, wherein the post-operative time period is between 4 and 21 days.

33. The method of claim 26, wherein the post-operative time period is between 4 and 7 days.

34. The method of claim 25, further comprising performing multiple single measurements per day in the post-operative time period.

35. The method of claim 25, further comprising performing a single measurement per day in the post-operative time period.

36. The method of claim 25, wherein the post-operative time period is the useful lifetime of the implanted medical device.

37. The method of claim 25, wherein the post-operative time period is 90 days.

38. A system for monitoring, in vivo, a concentration of a type of cell in a fluid around an implanted medical device in a body of a subject, comprising:
a monitoring device that generates an output signal indicative of an indicative of an amount of detected light in the fluid in response to light emitted into the fluid; and
an external device configured to establish a wireless communication connection with the monitoring device and to cause the monitoring device to transmit data corresponding to the output signal for at least a first single measurement to the external device over the wireless communication connection.

39. The system of claim 38 wherein the monitoring device comprises:
a light source having an output that emits the light having a narrow band of wavelengths into the fluid, wherein the band of wavelengths corresponds to a visible color of the type of cell;
a light sensor having a surface that detects light scattered by at least the cell in the fluid and an output that provides the output signal indicative of an amount of detected light, wherein the output signal of the light sensor has an active range in which the output signal varies substantially in response to detected light between an amount of detected light corresponding to a low concentration of the type of cell in the fluid and an amount of detected light corresponding to a high concentration of the type of cell in the fluid;
an electrical circuit including the light source and the light sensor and formed in a circuit board, the circuit board providing a relative spatial orientation of the light source and the light sensor such that a first direction of light emanating from the light source and a second direction orthogonal to and away from the surface of the light sensor are into the fluid and at substantially a same area in the fluid;
the electrical circuit further comprising a control circuit, the control circuit activating the light source and light sensor for a period of time for single measurement to cause the light sensor to generate an output signal; and
the electrical circuit further comprising a wireless transmitter configured to establish a communication connection with an external device and to transmit data corresponding to the generated output signal to the external device over the communication connection.

40. The system of claim 39, wherein the monitoring device is coated with a biocompatible, translucent, dielectric polymer at least on a surface exposed in the body of the subject.

41. The system of claim 38, wherein the external device is a general purpose computer executing an application to establish the wireless communication connection with the monitoring device.

42. The system of claim 38, wherein the external device further comprises a network interface for connection to a computer network and wherein the application transfers data received from the monitoring device to a server computer over the computer network.

43. The system of claim 38, wherein the external device is further configured to establishing a connection between the monitoring device and the external device to cause the monitoring device to transmit data corresponding to the output signal for a second single measurement to the external device.

44. The method of claim 43, wherein the first single measurement and the second single measurement are performed in a post-operative time period, after surgery to implant the implanted medical device.
